(19) United States
(12) Patent Application Publication
(10) Pub. No.: US 2025/0345565 A1
(43) Pub. Date: Nov. 13, 2025

(54) STEERABLE DEVICE FOR USE INSIDE OF A MAMMALIAN BODY

(71) Applicant: ETH ZÜRICH, Zürich (CH)

(72) Inventors: Sean Liam LYTTLE, Uster (CH); Roland DREYFUS, Zürich (CH); Alexandre MESOT, Zürich (CH); Bradley James NELSON, Zumikon (CH)

(73) Assignee: ETH ZÜRICH, Zürich (CH)

(21) Appl. No.: 18/870,567

(22) PCT Filed: Jun. 1, 2023
(86) PCT No.: PCT/EP2023/064650

§ 371 Date: Nov. 29, 2024

(30) Foreign Application Priority Data
Jun. 3, 2022 (EP) 22177147.0

(51) Int. Cl. A61M 25/01

(52) U.S. Cl. CPC A61M 25/0158

(57) ABSTRACT

A steerable device for use inside of a mammalian body, including an elongated element and a flexible device tip which includes a tubular section and a plurality of permanent magnetic elements disposed in the tubular section. Each magnetic element has an axial bore and the axial bores form an axial conduit in which a core wire extends. The axial bore extends from a first to a second axial end of the magnetic element opposed to the first axial end. First axial ends and second axial ends have a bevel allowing movement of the magnetic elements against one another, wherein the bevel of axial ends of adjacent magnetic elements is rounded with a different radius or flat with a different angle to the longitudinal axis to control bending of the device tip.

The invention relates to a steerable device for use inside of a mammalian body. The device may be a needle, guidewire, catheter, endoscope, or any other flexible device designed to be inserted into a mammalian body, especially lumens or cavities, for diagnostic or interventional purposes.

Many medical procedures require a medical instrument to be navigated to a specific location inside the body. Examples include deep brain stimulation, organ biopsies, targeted drug delivery, tumor removal, and many others. The insertion and navigation of such devices is achieved in a variety of ways depending on the application.

In the case of procedures inside body cavities, such as the abdomen or inside the bladder, or inside a vasculature system a flexible device is steered, usually by means of puller wires or rotating a pre-curved distal tip, while being pushed proximally. There is an inherent tradeoff between the device's ability to reach target sites and its ability to be advanced without buckling. Some proposed devices have a magnetic tip that allows a device to be more flexible and its trajectory to be more precisely controlled by way of a magnetic field.

U.S. Pat. No. 3,674,014 discloses a flexible catheter-tip, guidable by a magnetic field into selected arteries of the body, including a plurality of permanent magnetic tubular sections with ball-shaped ends arranged end-to-end. Each pair of adjacent ends is encased within a tubular link formed of non-magnetic material which provides a flexible, fluid-tight seal between the tubular sections. Each ball-shaped end has formed thereon a bevel to provide stability between adjacent sections, and therefore to the whole catheter-tip, at full bend. The diameter of such a device is limited by the fact that the diameter of the tubular sections with ball-shaped ends and of the tubular links are limited by their mechanical resistance.

US 2016/0089515 A1 discloses a wire guide for feeding a medical catheter through the body passage of a patient to a distant target site within the body having a variably flexible distal portion. The distal portion facilitates threading the guidewire in a tortuous path. The distal portion includes a cannula portion, a flexible portion, and a core wire. The flexible portion includes a plurality of spheroidal members that are displaced longitudinally at the distal end of a core wire and within a covering. The core wire is displaced internally passing through a first lumen within the cannula, and a second lumen defined by an aperture in each of the spheroidal members. The core wire is affixed to the distal-most member and has a proximal end that can be manipulated by a physician to compress or extend the plurality of members arranged in the flexible portion to control its flexibility and curvature. In addition, the members can be magnetically responsive to allow steering in a magnetic field. The diameter of such a device is limited by the fact that the core wire must extend over the whole length of the guidewire to be manipulated. Consequently, the reduction of the diameter of the core wire and inherently of the catheter is limited by the risk to break the core wire during manipulation.

Such devices allow a more precise navigation in the body. However, there is a need to reach target sites that are situated even further in body cavities or vasculature systems through acute bends at branch junctions in the body passages and lumens having smaller dimension with increasing distances.

It is an object of the present invention to provide a device for use inside of a mammalian body, which can be used in a reliable and reproducible manner in applications for diagnostic and/or interventional purposes requiring a great precision in the navigation to reach target sites through acute bends at branch junctions in the body passages and through lumens having smaller dimension, and which is not subject to the limitations of the devices listed above.

This objective is met according to the present invention by providing a device with the features of claims 1 and 15. Further advantageous embodiments of the invention are the subject of the dependent claims.

The steerable device can be a needle, guidewire, catheter, endoscope, or the like that is used inside of a mammalian body, in particular of a human, for example to inspect and/or operate. The steerable device may be used for inspecting and/or operating inside of organs (e.g. liver, lungs, kidney, brain, etc.), inside of a body cavity (e.g. abdomen, spinal cord, sinuses etc.), or in the vasculature. More generally, it is designed to reach target sites through acute bends in the body passages and lumens having small dimensions.

The steerable device comprises a flexible elongated element, which is elongated along a longitudinal axis and configured to be navigated through the mammalian body.

Further, the steerable device comprises a flexible device tip arranged at a distal end of the elongated element. The device tip is configured to be bent by way of an external magnetic field to allow steering of the elongated element.

An end of the steerable device opposed to the device tip, i.e. a proximal end of the steerable device, is designed to remain outside of the mammalian body for external manipulation for example by a physician or a robot.

In particular, the elongated element ends distally in the device tip. The distal tip may contain or carry measurement and/or actuating devices, such as a camera, sensors, ablation tip, needle, electrodes, etc. The device may contain an inner lumen that allows delivery of fluids, needles, coils, drugs, biopsy tools and the like to a target site.

The device tip comprises a tubular section and a plurality of permanent magnetic elements disposed in the tubular section end-to-end with adjacent ends of opposite polarity and their respective magnetic axes disposed along the longitudinal axis of the elongated element. This arrangement ensures that the magnetic elements attract each other in the longitudinal direction so that the plurality of magnetic elements form a stable structure in the tubular section. The tubular section is arranged and extends distally to the elongated element in the longitudinal direction. The tubular section can be in the form of a tube enveloping the plurality of magnetic elements, the tube being attached to the distal end of the elongated element or formed as a hollow extension of the elongated element, more specifically of an external envelope of the elongated element, in the longitudinal direction. The tubular section keeps the magnetic elements in the steerable device aligned in the longitudinal direction. Further, the magnetic force acting between magnetic elements ensures that the plurality of magnetic elements keeps its axial alignment in the tubular section in the absence of the magnetic field or returns to its axial alignment when the magnetic is switch off. Each magnetic element has an axial bore extending along its magnetic element longitudinal axis from a first axial end of the magnetic element to a second axial end of the magnetic element opposed to the first axial end, and the axial bores of the plurality of magnetic elements form an axial conduit through the plurality of magnetic elements. The intersection of the first axial end and the intersection of the second axial end with the axial bore define each time a bore edge. The first axial end of the magnetic element forms a free end of the magnetic element, and the second axial end of the magnetic element forms a free end of the magnetic element.

In addition, the steerable device comprises a core wire extending in the conduit at least through two magnetic elements of the plurality of magnetic elements. The core wire can be solid. Optionally, the core wire can also include a lumen. In this case, the core wire can be more flexible than a solid core wire. The core wire can flex between magnetic elements, i.e. at an interface between two consecutive magnetic elements, under the action of the magnetic field which allows the device tip to conform to the curvature of a path along which the steerable device must be moved in the mammalian body.

According to the invention, first axial ends and second axial ends have a bevel to allow for movement of the magnetic elements against one another and bending of the device tip.

The provision of bevels allows a movement of the magnetic elements relative to each other at the first axial ends and the second axial ends which movement can in turn be used to control or limit bending of the device.

In a preferred embodiment, the bevel is flat, as seen in a longitudinal section of the magnetic element, and forms a base angle with respect to the magnetic element longitudinal axis, wherein the bevel of an axial end and the bevel of a further axial end of a pair of adjacent ends of successive magnetic elements form a base angle and a further base angle, respectively, designed to allow bending of the device tip by a bending angle corresponding to the sum of the base angle and the further base angle. In this embodiment, the range of bending angles that can be achieved by the device tip can be defined for each pair of adjacent ends of successive magnetic elements. This allows a precise control of the bending of the device tip, namely at the level of two successive magnetic elements. Further, flat bevels provide for stiffness necessary to avoid buckling the steerable device when reaching a target site. Indeed, flat bevels reduce the risk that magnetic elements flip over each other or that ends of magnetic elements slip over each other when maximal bending is reached.

In a preferred embodiment, first axial ends and second axial ends have the same bevel to allow the same bend radius between adjacent magnetic elements for the ease of manipulation and manufacturing. The bevels can be rounded or flat, as seen in a longitudinal section of the magnetic elements.

In a preferred embodiment, first axial ends and second axial ends have a different bevel to allow a progressive increase or reduction of bend radius between adjacent magnetic elements from proximal to distal. The bevels can be rounded or flat, as seen in a longitudinal section of the magnetic elements.

Further preferred embodiments having a flat bevel are disclosed in a later section.

In a preferred embodiment, the bevel is rounded, as seen in a longitudinal section of the magnetic element, to allow bending of the device tip.

Further preferred embodiments having a rounded bevel are also disclosed in a later section.

In addition, it is also conceivable to provide for embodiments in which magnetic elements have a flat bevel at the first axial end and a rounded bevel at the second axial end.

It is also conceivable to have a first plurality of successive magnetic elements which first axial ends and second axial ends have the same first bevel and a second plurality of successive magnetic elements which first axial ends and second axial ends have the same second bevel, the first bevel being different to the second bevel. This embodiment allows to have sections with different bend radius which may be useful for certain types of operations. The first bevel and the second bevel can be rounded or flat.

In a preferred embodiment, a first end of the core wire is retained at a first end of the tubular section and a second end of the core wire opposed to the first end of the core wire is arranged in the conduit and is free, to allow an axial displacement of the plurality of magnetic elements relative to the core wire when the device tip is bent. The arrangement allows acute bends of the device tip because the core wire keeps the magnetic elements aligned when the device tip is bent in addition to the interaction between the magnetic elements, while at the same time the flexibility of the device tip, i.e. the curvature of the device tip, can be configured by way of the flexibility of the core wire used in the steerable device. Therefore, it is not necessary that an end of the core wire extends up to the proximal end of the steerable device to be manipulated by a physician that compress or extend the arrangement of magnetic members in the tubular section to control the flexibility and curvature of the device tip. Further, this arrangement supports a reduction of the diameter of the steerable device.

In a preferred embodiment, the first end of the core wire is retained by a retaining portion arranged proximally to the device tip in the elongated element and extends through the elongated element and the second end of the core wire opposed to the first end of the core wire is arranged in the conduit and is free, to allow an axial displacement of the plurality of magnetic elements relative to the core wire when the device tip is bent. In the case of first end formed as a knot, the feature first end is to be understood as the knot from which a short piece of core wire can extend proximally.

It is also conceivable that the core wire extends proximally to the device tip over a portion of the steerable device in direction to proximal or till a proximal end of the steerable device, wherein the core wire is fixed by a retaining portion arranged proximally to the device tip in the elongated element, the retaining portion being arranged at an intermediary point of the core wire situated between the proximal end and the distal end of the core wire. The arrangement allows also acute bends of the device tip because the core wire keeps the magnetic elements aligned when the device tip is bent in addition to the interaction between the magnetic elements. It allows also to increase the stiffness of the elongated element over the portion of the core wire that is extending in the elongated element outside of the tubular section.

In a preferred embodiment, the plurality of magnetic elements extends over the whole axial length of the tubular section. In other words, the tubular section is filled with magnetic elements from the first end of the tubular section to a second end of the tubular section opposed to the first end of the tubular section. This embodiment allows the use of the full length of the tubular section to steer the steerable device.

The term “free” in relation to an end of the core wire means that the end of the core wire can move axially but is limited radially in its movement by an inner surface of the conduit.

In the present disclosure, the term “proximal” refers to the direction or the side oriented towards the operator of the steerable device, for example a physician. Further, the term “distal” refers to the direction or the side oriented towards the patient.

In a preferred embodiment, the first end of the core wire is the proximal end of the core wire, the first end of the tubular section is the proximal end of the tubular section and the second end of the core wire is the distal end of the core wire. In other words, a proximal end of the core wire forms a retained end and is retained at a proximal end of the tubular section and a distal end of the core wire forms the free end and is free in the conduit, to allow an axial displacement of the plurality of magnetic elements relative to the core wire when the device tip is bent. The arrangement allows an increasing bend in the direction from proximal to distal, i.e. a bend radius becoming smaller from proximal to distal, because the magnetic elements arranged proximally in the tubular section are kept in place or are limited in their displacement while the magnetic elements arranged distally in the tubular section can have a larger relative displacement with respect to the core wire.

In a preferred embodiment, the first end of the core wire is the distal end of the core wire, the first end of the tubular section is the distal end of the tubular section and the second end of the core wire is the proximal end of the core wire. In other words, a distal end of the core wire forms a retained end and is retained at a distal end of the tubular section and a proximal end of the core wire forms the free end and is free in the conduit, to allow an axial displacement of the plurality of magnetic elements relative to the core wire when the device tip is bent. The arrangement allows an increasing bend in the direction from distal to proximal, i.e. a bend radius becoming smaller from distal to proximal, because the magnetic elements arranged distally in the tubular section are kept in place or are limited in their displacement while the magnetic elements arranged proximally in the tubular section can have a larger relative displacement with respect to the core wire.

In a preferred embodiment, the distal end of the tubular element forms the end of the steerable device to allow a precise steering of the steerable device.

In a preferred embodiment, the length of the core wire is equal to the axial length of the plurality of magnetic elements. The length of the core wire may also include the core wire portion, if any, used to retain the core wire.

In an embodiment in which the core wire is retained at a first end of the tubular section and the second end is free in the conduit formed by the plurality of magnetic elements arranged in said tubular section, it follows that the core wire can have approximately the same length or can be shorter than the length of the tubular section. By construction, the core wire is shorter than the elongated element or the steerable device. In particular, the core wire is free from a proximal end extending outside of the elongated element or the steerable device, for example to allow direct manipulation of the core wire by an operator.

In a non-deformed state that can be a resting state, the length of the core wire can be equal to or shorter than the axial length of the plurality of magnetic elements. In a deformed state, it results that the free end of the core wire is displaced into the magnetic element in which it is lodged, i.e. in the direction toward the retained end, as seen relative to the magnetic element in which it is lodged.

The number of permanent magnetic elements and their respective length can be chosen to allow the device tip to bend and conform optimally to the curvature of the path along which the steerable device must be moved in the mammalian body. The choice depends also on the properties of the steerable device required for the operation. In a preferred embodiment, the plurality of permanent magnetic elements comprises 3 to 100 magnetic elements. Embodiments with more than 100 magnetic elements can also be conceived. In a more preferred embodiment, the plurality of permanent magnetic elements comprises 3 to 20 magnetic elements to allow an optimal bend of the device tip and a simpler manufacturing at the same.

In a preferred embodiment, each magnetic elements of the plurality of magnetic elements have an axial symmetry around their magnetic axis. This allows a simple control of the device tip in a magnetic field.

In a preferred embodiment, the magnetic elements of the plurality of magnetic elements have the same axial length. This allows a homogeneous bending with an approximately continuous bend radius of the device tip in the magnetic field. The control of the steerable device is further improved.

In a preferred embodiment, the core wire is made of a plurality of strands, wherein the strands of the plurality of strands can be retained at the first end of the core wire and free at the second end of the core wire. As a result, the flexibility of the core wire can be designed with more degrees of freedom in view of the physical properties of the different strands. The curvature of the device tip can be configured by way of the flexibility of the strands chosen for the core wire.

In a preferred embodiment, the strands are made of the same material to form a core wire with homogeneous properties so that the steerable device can be easily controlled. It is also possible to provide for a core wire having at least one strand made of a different material, for example to ensure a minimum resistance of the tubular section to strains when it is deformed.

The core wire made of a plurality of strands, also referred to as stranded core wire, has an outer diameter defined as the diameter of the cylindrical virtual surface enveloping the plurality of strands and contacting the radially outmost surfaces of the strands.

In a more preferred embodiment, the core wire is made of a plurality of strands extending parallel to each other along the longitudinal axis, wherein the strands of the plurality of strands can be retained at the first end of the core wire and free at the second end of the core wire. This arrangement allows a simple manufacturing of the device tip.

In a more preferred embodiment, the core wire is made of a plurality of strands bundled along the longitudinal axis, wherein the strands of the plurality of strands can be retained at the first end of the core wire and free at the second end of the core wire. For example, the strands can be bundled in a helical manner and wound around each other. Bundled strands have the advantage that the core wire can be further extended and consequently can show additional flexibility because the strands can unwind slightly while the device tip is bent.

In a preferred embodiment, the plurality of strands comprises two to seven strands. In particular, an arrangement with three or seven strands allows a symmetrical construction that provide for an easy bending control. Preferably three strands are used to reduce the diameter of the core wire. The number of strands can also be used as a parameter to tune the flexibility of the core wire.

In a preferred embodiment, the strands of the plurality of strands have different lengths. The resulting effect is that the flexibility of the core wire is different along the longitudinal direction and consequently the bend radius that can be obtained is different along the longitudinal direction. The flexibility is the lowest in a portion of the core wire which cross section comprises all strands of the plurality of strands, typically at the first end or in the region of the first end in which the plurality of strands is retained. The flexibility increases in a further portion of the core wire which cross section comprises at least one strand less, i.e. when the further portion is arranged distally to the shortest strand of the plurality of strands.

In embodiments in which the core wire is made of a plurality of strands, each strand has a free end. The length of the core wire is defined as the length of the longest strand. The end of the core wire is defined at the end of the longest strand.

In a preferred embodiment, the plurality of strands have each a length corresponding to a multiple of the length of the magnetic elements. Preferably, at least one strand extending over the full axial length of the plurality of magnetic elements. The at least one strand extending over the whole axial length keeps the magnetic elements aligned when the device tip is bent. In this embodiment, in the process of deforming to the deformed state for example under the action of the magnetic field, each strand is displaced each time into the magnetic element in which it is lodged in the non-deformed state in the direction to the retained end, as seen relative to the magnetic element, and is not displaced from a magnetic element to an adjacent magnetic element. As a result, in the process of deforming, the flexibility remains constant at the interface between two consecutive magnetic elements. A jump in the flexibility can be avoided while deforming as it would be the case each time a strand is displaced from a magnetic element to an adjacent magnetic element. The control of the steerable device is improved.

In a preferred embodiment, the outer diameter of the core wire is substantially the same as an interior diameter of the conduit to keep the magnetic elements aligned in the longitudinal direction.

The term “substantially the same” means that the outer diameter of the core wire is the same within the manufacturing tolerance such that the tolerance allows a displacement of the core wire within the conduit. In particular, friction between the core wire and the magnetic elements must be kept low to ensure that the device tip springs back to its non-deformed state in the absence of magnetic field and does slightly deformed.

In a preferred embodiment, the outer diameter of the core wire at its first end is preferably substantially the same as the interior diameter of the conduit and decreases in the direction to the second end. In this embodiment, the flexibility of the device tip increases from proximal to distal to allow sharp bend.

In a preferred embodiment, the steerable device comprises a retaining portion arranged at the first end of the tubular section to retain the first end of the core wire at the first end of the tubular section. The retaining portion can be a part of the tubular section configured to retain the first end of the core wire at the first end of the tubular section. In a preferred embodiment, the retaining portion and the tubular section are formed in one piece to reduce production and assembly costs.

In a preferred embodiment, the steerable device comprises the retaining portion arranged at the first end of the tubular section, preferably in the tubular section, the retaining portion having a passage extending axially from the side facing away from the second end of the tubular section through the retaining portion to a side of the retaining portion facing the second end of the tubular section. The passage allows to insert the core wire to retain the first end of the core wire on the side facing away from the second end of the tubular section.

In a preferred embodiment, the steerable device comprises the retaining portion arranged at the first end of the tubular section, preferably in the tubular section, the retaining portion having a cavity on its side facing away from the second end of the tubular section and having the passage extending axially from the cavity through the retaining portion to the side of the retaining portion facing the second end of the tubular section. The cavity allows to protect the first end of the core wire when it is retained in the cavity and limits the movements of the first end of the core wire.

In a preferred embodiment, the first end of the core wire has a thickening, preferably in the form of a bead, wherein the passage is designed to retain axially the thickening on the side facing away from the second end of the tubular section, when the core wire is inserted in the passage. This embodiment has the advantage that the first end of the core wire is maintained in a position along the longitudinal axis in a manner simple to assemble.

In a preferred embodiment, the steerable device comprises a retaining portion arranged at the first end of the tubular section, preferably in the tubular section, the retaining portion having a cavity on its side facing away from the second end of the tubular section and having a passage extending axially from the cavity through the retaining portion to a side of the retaining portion facing the second end of the tubular section. Further, the first end of the core wire has a thickening, preferably in the form of a bead, wherein the cavity is designed to receive the thickening and the passage is designed to retain axially the thickening in the cavity when the core wire is inserted in the passage. This embodiment has the advantage that the first end of the core wire is maintained in a position along the longitudinal axis that can move axially in a manner limited by the cavity in which the core wire is retained.

In a preferred embodiment, in which the core wire is made of a plurality of strands, each strand can have a first end having a thickening, preferably in the form of a bead, wherein the passage is designed to retain axially the plurality of thickenings when the core wire is inserted in the passage.

In a preferred embodiment, in which the core wire is made of a plurality of strands, each strand can have a first end having a thickening, preferably in the form of a bead, wherein the cavity is designed to receive the plurality of thickenings and the passage is designed to retain axially the plurality of thickenings in the cavity when the core wire is inserted in the passage.

In a preferred embodiment in which the first end of the core wire is the proximal end of the core wire, the first end of the tubular section is the proximal end of the tubular section, and the second end of the core wire is the distal end of the core wire, the steerable device comprises a retaining portion in the form of a connection piece. The connection piece is inserted at least partially at the distal end of the elongated element in the elongated element, preferably in a non-detachable manner, is arranged at the proximal end of the tubular section and delimits, in its inserted position, a cavity on its side facing the elongated element. Further, the connection piece has an opening extending axially from the cavity to the tubular section. Further, the proximal end of the core wire has a thickening, preferably in the form of a bead, wherein the cavity is designed to receive the thickening and the opening is designed to retain axially the thickening in the cavity when the core wire is inserted in the passage.

Any other form of thickening can also be used, for example also in the form of a knot or a crimp, as long as it is designed to retain axially the core wire.

In a preferred embodiment, the steerable device comprises a retaining portion arranged at the first end of the tubular section, preferably in the tubular section, the core wire being glued to the retaining portion or melted with the retaining portion. In both embodiments, the core wire is retained in a fixed manner that is easy to manufacture at lower costs.

In a preferred embodiment, each magnetic element of the plurality of magnetic elements is made of two magnetic parts that are symmetrical to each other with respect to a symmetry plane cutting the magnetic axis perpendicularly at a middle point of the magnetic element as seen in the longitudinal direction, and are disposed end-to-end with adjacent ends of opposite polarity. In other words, each magnetic element is made of a first magnetic part and a second magnetic part, the first magnetic part and the second magnetic part having a flat end extending in a symmetry plane perpendicular to the magnetic axis, wherein the first magnetic part and the flat end of the second magnetic part are arranged adjacent to each other to form the magnetic element. The free end of the first magnetic part and the free end of the second magnetic part opposed to their respective flat end can have a bevel or have a form suitable to allow for movement of the magnetic elements against each other. This embodiment is easy to manufacture because the flat end can be taken as reference surface to manufacture the free end of the magnetic parts, in particular when the free end has a bevel.

In a preferred embodiment, a most proximal magnetic element of the plurality of magnetic elements is made of single magnetic part having the flat end facing the elongated element to provide for an interface between the elongated element and the device tip that has a simple design and ensure a reliable transmission of movements of the elongated element to the device tip.

In a preferred embodiment, the first axial ends of the plurality of magnetic elements and the second axial ends of the plurality of magnetic elements have each a bevel to form convex first axial ends and second axial ends allowing for movement of the magnetic elements against one another.

In a preferred embodiment, the bevel has a bevel edge starting each time at a radial distance from the bore edge of the axial bore. This embodiment ensures that the interface between two successive magnetic elements has a surface of contact that allows a distribution of the contact forces minimizing mechanical strains that could result in damaging the interface between the two successive magnetic elements.

In a preferred embodiment, the bevel is flat, as seen in a longitudinal section of the magnetic element, and forms a base angle with respect to the magnetic element longitudinal axis, wherein the bevel of a first pair of adjacent ends of successive magnetic elements has a same first base angle, the bevel of a second pair of adjacent ends of successive magnetic elements has a same second base angle, and the first base angle is different from the second base angle. The flat bevel is formed such that the profile of the bevel follows a straight line, as seen in a longitudinal section of the magnetic element, and the base angle is defined as the acute angle formed between the straight line and the magnetic element longitudinal axis. This configuration allows a different bend radius of the device tip along the longitudinal axis.

In a more preferred embodiment, the first pair of adjacent ends is arranged proximally to the second pair of adjacent ends and the first base angle is smaller than the second base angle. This configuration allows a bending angle between the first pair of adjacent ends that is smaller than a bending angle between the second pair of adjacent ends, i.e. a bend radius of the device tip smaller at the interface between the first pair of adjacent ends than at the interface between the second pair of adjacent ends, i.e. the bend radius of the device tip increases from proximal to distal.

In a more preferred embodiment, the plurality of base angles are in the range between 30° to 88°. This range of angles allows the deformation of the device tip in the form of a sequence of segments—each corresponding to a magnetic element—having an angle relative to each other of 60° to 176°. This range allows a bend radius of the device tip that is suitable to conform to most path curvatures along which the steerable device must be moved in the mammalian body.

In an even more preferred embodiment, the plurality of base angles have the same value in the range between 45° to 80°. Simulations have shown optimum bend radius in view of path curvatures along which the steerable device must be moved in the mammalian body.

In a preferred embodiment, the bevel is rounded, as seen in a longitudinal section of the magnetic element, wherein the bevel of a first pair of adjacent ends of magnetic elements has a same first curvature radius, the bevel of a second pair of adjacent ends of magnetic elements has a same second curvature radius, and the first curvature radius is different from the second curvature radius. The rounded bevel is convex, i.e. curves outwards with respect to the magnetic element, and its profile follows a curved line. This configuration allows a different bend radius of the device tip along the longitudinal axis.

In a preferred embodiment, the rounded bevel is in the form of a circular arc or in the form of an elliptical arc. As a result, the end of the magnetic element is spherical or the end of the magnetic element is elliptical and forms a portion of an ellipsoid, respectively.

In a more preferred embodiment, the first pair of adjacent ends is arranged proximally to the second pair of adjacent ends and the first curvature radius is smaller than the second curvature radius. This configuration allows a bending angle between the first pair of adjacent ends that is greater than a bending angle between the second pair of adjacent ends, i.e. a bend radius of the device tip smaller at the interface between the first pair of adjacent ends than at the interface between the second pair of adjacent ends, i.e. the bend radius of the device tip increases from proximal to distal.

Rounded portions and flat portions can be combined to optimize the form of a bevel and accordingly the bend capacity of the steerable device.

A steerable device for use inside of a mammalian body is also disclosed, comprising a flexible elongated element, elongated along a longitudinal axis, and configured to be navigated through the mammalian body. Further, the steerable device comprises a flexible device tip arranged at the distal end of the elongated element and configured for allowing steering of the elongated element by way of an external magnetic field. Moreover, the device tip comprises a tubular section and a plurality of permanent magnetic elements disposed in the tubular section end-to-end with adjacent ends of opposite polarity and their respective magnetic axes disposed along the longitudinal axis, wherein each magnetic element has an axial bore extending along its magnetic element longitudinal axis from a first axial end of the magnetic element to a second axial end of the magnetic element opposed to the first axial end, and the axial bores of the plurality of magnetic elements form an axial conduit through the plurality of magnetic elements. The steerable device also comprises a core wire extending in the conduit through the plurality of magnetic elements.

In a preferred embodiment, a first end of the core wire is retained at a first end of the tubular section and a second end of the core wire opposed to the first end is retained at a second end of the tubular section, wherein the core wire is designed to allow an axial displacement of the plurality of magnetic elements relative to the core wire when the tip is bent. The arrangement allows acute bends of the device tip because the core wire keeps the magnetic elements aligned when the device tip is bent, while at the same time the flexibility of the device tip, i.e. the curvature of the device tip, can be configured by way of the core wire used in the steerable device. Therefore, it is not necessary that an end of the core wire extends up to the proximal end of the steerable device to be manipulated by a physician that compress or extend the arrangement of magnetic members in the tubular section to control the flexibility and curvature of the device tip.

In a preferred embodiment, the steerable device comprises a retaining portion arranged at the first end of the tubular section to retain the first end of the core wire at the first end of the tubular section and a further retaining portion arranged at the second end of the tubular section to retain the second end of the core wire at the second end of the tubular section. The retaining portion and the further retaining portion can be a part of the tubular section configured to retain the first end of the core wire at the first end of the tubular section and the second end of the core wire at the second end of the tubular section.

In a preferred embodiment, the retaining portion, the further retaining portion, and the tubular section are formed in one piece to reduce production and assembly costs.

In a preferred embodiment, the first end of the core wire is retained proximally to the device tip in the elongated element, preferably by a retaining portion arranged in the elongated element, and extends through the elongated element, and the second end of the core wire opposed to the first end of the core wire is retained at a second end of the tubular section, preferably by a further retaining portion.

In a preferred embodiment, the core wire is designed in such a way that it is deformable in an elastic manner to allow the axial displacement of the plurality of magnetic elements relative to the core wire when the tip is bent, wherein the first end of the core wire and the second end of the core wire are retained in a fixed manner. In this embodiment, the flexibility of the device tip, i.e. the curvature of the device tip, can be configured by way of the elasticity of the core wire used in the steerable device, i.e. by the difference in length between the core wire in its deformed state and the plurality of magnetic elements. Deformation in an elastic manner of the core wire allows an extension of its length in the longitudinal direction under the influence of a force exerted resulting from bending the device tip. When that force is removed the core wire returns to its original length and shape. The core wire is designed in such a way that it is deformable in an elastic manner in the longitudinal direction.

In a preferred embodiment, the core wire is designed in such a way that the length of the core wire is longer than the axial length of the plurality of magnetic elements, wherein the first end of the core wire and/or the second end of the core wire are retained with a play designed to allow the axial displacement of the plurality of magnetic elements relative to the core wire when the tip is bent. In this embodiment, the flexibility of the device tip, i.e. the curvature of the device tip, can be configured by way of the difference in length between the core wire and the plurality of magnetic elements.

In a preferred embodiment, the first end of the core wire is retained proximally to the device tip in the elongated element, preferably by a retaining portion arranged in the elongated element, and extends through the elongated element, and the second end of the core wire opposed to the first end of the core wire is retained at a second end of the tubular section, preferably by a further retaining portion.

In a preferred embodiment, the difference in length between the core wire and the plurality of magnetic elements is approximately 2% to 10% to allow a longitudinal displacement of the core wire. This embodiment allows acute bends of the device tip at branch junctions in the body passages.

The preferred features of the steerable device disclosed in relation to the embodiments in which the first end of the core wire is retained at the first end of the tubular section and the second end of the core wire opposed to the first end of the core wire is arranged in the conduit and is free described above also apply to the embodiments in which the first end of the core wire is retained at the first end of the tubular section and the second end of the core wire opposed to the first end is retained at the second end of the tubular section without or with a play, with the same advantages and effects, with the exception of the features related specifically to the free end of the core wire as disclosed in relation to first aspect of the invention.

It follows in particular that for the embodiments in which the first end of the core wire is retained at the first end of the tubular section and the second end of the core wire opposed to the first end is retained at the second end of the tubular section without or with a play, the steerable device can comprise a retaining portion arranged at the first end of the tubular section to retain the first end of the core wire at the first end of the tubular section and a further retaining portion arranged at the second end of the tubular section to retain the second end of the core wire at the second end of the tubular section.

In a preferred embodiment, the steerable device comprises the retaining portion arranged at the first end of the tubular section, preferably in the tubular section, and the further retaining portion arranged at the second end of the tubular section, preferably in the tubular section. The retaining portion has a passage extending axially from the side facing away from the second end of the tubular section through the retaining portion to a side of the retaining portion facing the second end of the tubular section. The further retaining portion has a further passage extending axially from the side facing away from the first end of the tubular section through the further retaining portion to a side of the further retaining portion facing the first end of the tubular section. The passage and the further passage allow to insert the core wire to retain the first end of the core wire on the side facing away from the second end of the tubular section and to retain the second end of the core wire on the side facing away from the first end of the tubular section, respectively.

In a preferred embodiment, the retaining portion has a cavity on its side facing away from the second end of the tubular section, the passage extending axially from the cavity through the retaining portion, and the further retaining portion has a cavity on its side facing away from the first end of the tubular, the further passage extending axially from the cavity through the retaining portion. The cavities allow to protect the first end and the second end of the core wire when they are retained in the respective cavity and limit the movements of the first end and the second end of the core wire.

In a preferred embodiment, the first end of the core wire has a thickening, preferably in the form of a bead, wherein the passage is designed to retain axially the thickening on the side facing away from the second end of the tubular section, when the core wire is inserted in the passage. Any other form of thickening can also be used, for example also in the form of a knot or a crimp, if it is designed to retain axially the core wire. Further, the second end of the core wire has a thickening, preferably in the form of a bead, wherein the further passage is designed to retain axially the thickening on the side facing away from the first end of the tubular section, when the core wire is inserted in the further passage. This embodiment has the advantage that the first end and the second end of the core wire are maintained in a position along the longitudinal axis in a manner simple to assemble. Further, the cavities limit axially the movement of the core wire when it is retained.

### DESCRIPTION OF THE FIGURES

FIG. 1 shows a schematic perspective of a distal portion of a steerable device according to a first embodiment of the invention;

FIG. 2 shows a schematic longitudinal cross-section of a device tip of the steerable device according to a second embodiment of the invention;

FIG. 3 shows a schematic detailed cross-section of the region annotated with B in the embodiment of FIG. 2;

FIG. 4 shows a schematic detailed cross-section of the region annotated with C in the embodiment of FIG. 2;

FIG. 5 shows a schematic detailed cross-section of the region annotated with D in the embodiment of FIG. 2;

FIG. 6 shows a schematic side view of the device tip of the steerable device according to a third embodiment of the invention;

FIG. 7 shows a schematic side view of the regions annotated with 1, 2 and 3 in the embodiment of FIG. 6;

FIG. 8 shows a schematic longitudinal cross-section of a device tip of the steerable device according to a fourth embodiment of the invention; and

FIG. 9 shows a schematic longitudinal cross-section of a device tip of the steerable device according to a fifth embodiment of the invention.

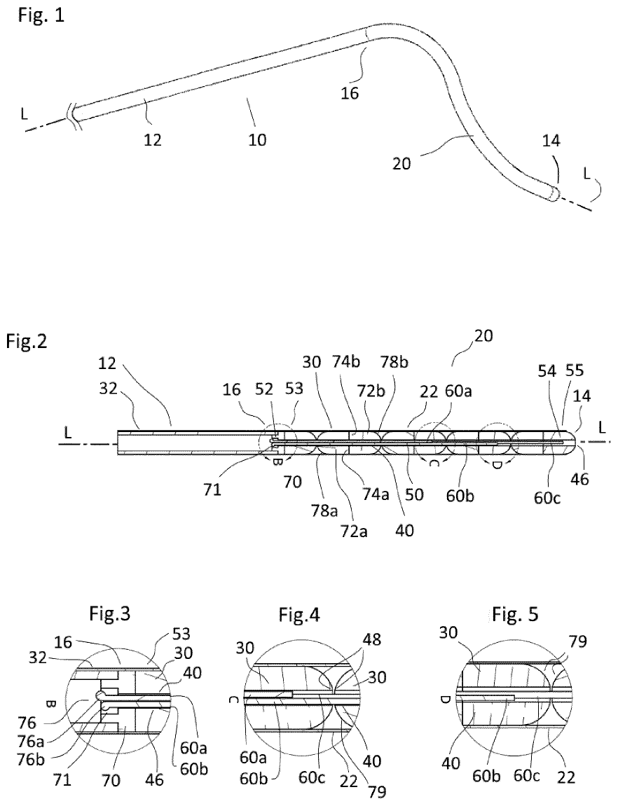

A first embodiment of the steerable device 10 according to the invention is configured as a catheter as illustrated in FIG. 1.

The steerable device 10 has an at least approximately rotationally symmetrical structure in relation to its longitudinal axis with a narrow circular section in proportion to its length. It comprises a flexible elongated element 12 in the form of an elongate shaft, whose longitudinal axis corresponds to the longitudinal axis L of the steerable device 10, having a circular section and extending over the length of the steerable device 10. The steerable device 10 has a proximal end that is not represented in FIG. 1 and a distal end 14 on the side of an operation site.

Further, the steerable device 10 comprises a flexible device tip 20 arranged at a distal end 16 of the elongated element 12. The device tip 20 is configured to be bent by way of an external magnetic field to allow steering of the elongated element. In particular, the elongated element 12 ends distally in the device tip 20. The device tip 20 is bent under the action of a magnetic field in a deformed state.

In a second embodiment of the steerable device in a non-deformed state as illustrated in FIG. 2 as well as in FIGS. 3, 4 and 5 more specifically, the device tip 20 comprises a tubular section 22 and a plurality of permanent magnetic elements 30 disposed in the tubular section 22 end-to-end with adjacent ends of opposite polarity and their respective magnetic axes disposed along the longitudinal axis L of the elongated element. In the embodiment disclosed in FIG. 2, there are five magnetic elements. The magnetic elements 30 attract each other in the longitudinal direction L so that the plurality of magnetic elements 30 form a stable structure in the tubular section 22.

The tubular section 22 is formed as a tube enveloping the plurality of magnetic elements 30 and extending distally in the longitudinal direction from the elongated element 12, flush with an external envelope 32 of the elongated element. The tubular section 22 keeps the magnetic elements 30 aligned in the steerable device 10 in the longitudinal direction L together with the magnetic force acting between magnetic elements.

Each magnetic element 30 has an axial bore 40 extending along its magnetic element longitudinal axis from a first axial end of the magnetic element to a second axial end of the magnetic element opposed to the first axial end. The axial bores 40 of the plurality of magnetic elements form an axial conduit 46 through the plurality of magnetic elements. The intersection of the first axial end and the intersection of the second axial end with the axial bore define each time a bore edge 48. Each magnetic elements of the plurality of magnetic elements has an axial symmetry around their magnetic axis.

In addition, the steerable device 10 comprises a core wire 50 extending in the conduit 46 at least through two magnetic elements 30 of the plurality of magnetic elements. The core wire 50 can flex between magnetic elements, i.e. at an interface between two consecutive magnetic elements, under the action of the magnetic field which allows the device tip 20 to conform to the curvature of a path along which the steerable device 10 must be moved in the mammalian body.

In the embodiment disclosed in FIG. 2, a first end of the core wire is retained at a first end of the tubular section 22 and a second end of the core wire opposed to the first end is arranged in the conduit 46. The second end is free to allow an axial displacement of the plurality of magnetic elements 50 relative to the core wire 50 when the device tip 20 is bent. Embodiments in which the first end of the core wire is retained in the elongated element proximal to the tubular section are also conceivable.

The tubular section 22 is filled with magnetic elements 50 from the first end of the tubular section to a second end of the tubular section opposed to the first end of the tubular section, i.e. over its whole axial length. In the embodiment shown, the tubular section contains four magnetic elements 50 and proximal to the four magnetic elements a magnetic part having the form of the half of a magnetic element 50.

Presently, the first end of the core wire 50 is the proximal end 52 of the core wire, the first end of the tubular section is the proximal end 53 of the tubular section, the second end of the core wire is the distal end 54 of the core wire and the second end of the tubular section is the distal end 55 of the tubular section.

The core wire 50 is made of a plurality of strands, wherein the strands 60a, 60b, 60c of the plurality of strands can be retained at the first end of the core wire, i.e. the proximal end 52 of the core wire in the present embodiment, and free at the second end of the core wire, i.e. the distal end 54 of the core wire in the present embodiment.

In FIG. 2, the core wire has three strands 60. The strands 60a, 60b, 60c of the plurality of strands have different lengths. In the embodiment of FIG. 2 in which the core wire 50 has three strands, a first strand 60a extends up to a second magnetic element, a second strand 60b extends up to a second magnetic element and a third strand 60c, the longest strand, extends over 100% of the length of the plurality of magnetic elements, here up to a fourth magnetic element. The resulting effect is that the flexibility of the core wire is different along the longitudinal direction and consequently the bend radius that can be obtained is different along the longitudinal direction after 50%, 75% and 100% of the core wire length.

The flexibility is the lowest, i.e. the bend radius is the larger, in a first portion of the core wire which comprises all—here in FIG. 2 three—strands of the plurality of strands. The first portion extends from the proximal end 52 of the core wire 50, in which the plurality of strands 60a, 60b, 60c are retained, up to the end of the first strand 60a. The flexibility increases in a second portion of the core wire 50 extending distally to the first portion up to the end of the second strand 60b which comprises only two strands. The flexibility further increases in a third portion of the core wire 50 extending distally to the second portion up to the end of the third strand 60c and in which there is only one strand left. The transition from the first portion to the second portion is illustrated in FIG. 4. The transition from the second portion to the third portion is illustrated in FIG. 5.

As illustrated in FIG. 2 and FIG. 3, the steerable device 10 comprises a retaining portion 70 arranged in a non-detachable manner at the first end of the tubular section 22, here at the proximal end 53 of the tubular section, in the tubular section. The retaining portion 70 has a cavity 71 on its side facing away from the second end of the tubular section, i.e. on its side facing away from the distal end 55 of the tubular section, and having a passage 74 extending axially from the cavity 71 through the retaining portion 70 to a side of the retaining portion facing the second end 55 of the tubular section.

Further, the first end of the core wire has a thickening 76 in the form of a plurality of beads 76a, 76b, each bead being formed at the first end, here the proximal end, of a strand of the plurality of strands. The cavity 71 is designed to receive the thickening 76 and the passage 74 is designed to retain axially the thickening in the cavity when the core wire is inserted in the passage.

Each magnetic element 30 of the plurality of magnetic elements is made of two magnetic parts 72a and 72b that are symmetrical to each other with respect to a symmetry plane cutting the magnetic axis perpendicularly at a middle point of the magnetic element as seen in the longitudinal direction. The magnetic parts are disposed end-to-end with adjacent ends of opposite polarity. In other words, each magnetic element is made of a first magnetic part 72a and a second magnetic part 72b, the first magnetic part 72a and the second magnetic part 72b having each a flat end extending in the symmetry plane, wherein the flat end 74a of the first magnetic part 72a and the flat end 74b of the second magnetic part are 72b arranged adjacent to each other to form the magnetic element 30.

As can be seen in FIG. 2, the most proximal magnetic element of the plurality of magnetic elements is made of single magnetic part having the flat end facing the elongated element to provide for a simple interface between the elongated element and the device tip.

The free end 78a of the first magnetic part and the free end 78b of the second magnetic part opposed to their respective flat end have a bevel 79 to allow for movement of the magnetic elements 30 against each other. In the present embodiment, the bevel 79 is each time rounded, as seen in a longitudinal section of the magnetic element, wherein the bevel has a same curvature radius for all magnetic elements of the plurality of magnetic elements. This configuration allows a cost-effective manufacturing of the steerable device.

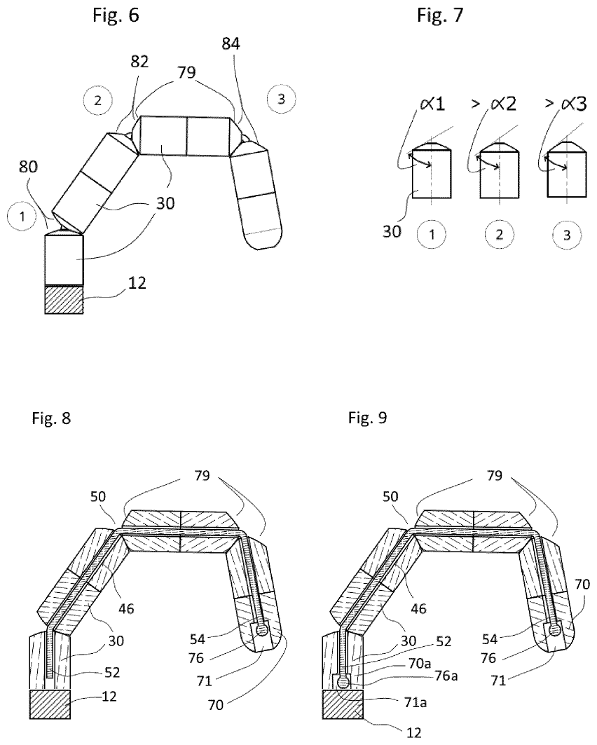

The device tip of the steerable device according to a third embodiment disclosed in FIG. 6 is represented as a side view without the tubular section, i.e. without the tube enveloping the plurality of magnetic 1 elements. This embodiment is essentially the same as the second embodiment disclosed in FIG. 2 and differentiates by the arrangement of the bevels at the free ends of the magnetic elements.

The bevel 79 of the free end of the magnetic elements in the third embodiment is flat and forms a base angle α (alpha) with respect to their respective magnetic element longitudinal axis L. Presently, seen from proximal to distal, the bevel 79 of a first pair 80 of adjacent free ends of magnetic elements has a same first base angle α1 (alpha 1), the bevel 79 of a second pair 82 of adjacent free ends of magnetic elements has a same second base angle α2 (alpha 2), and the bevel 79 of a third pair 84 of adjacent free ends of magnetic elements has a same third base angle α3 (alpha 3), wherein a1<α2>α3, i.e. the base angle increases from one pair of adjacent free ends to the next pair of adjacent free ends from proximal to distal.

The definition of the base angles α1, α2 and α3 corresponding to the pairs of adjacent free ends of the magnetic elements represented in FIG. 6 is represented schematically in FIG. 7.

As a result of this arrangement, the bend radius decreases from proximal to distal and the device tip can be bent more sharply from proximal to distal.

A fourth embodiment of the steerable device is disclosed in FIG. 8, of which only a portion of the elongated element 12 and the arrangement of the plurality of the magnetic elements without the tubular section is represented, i.e. without the tube enveloping the plurality of magnetic elements. A first end of the core wire is retained in a fixed manner in a retaining portion 70 at a first end of the tubular section and a second end of the core wire opposed to the first end is arranged in the conduit 46. The second end is free to allow an axial displacement of the plurality of magnetic elements 50 relative to the core wire 50 when the device tip 20 is bent.

Presently, the first end of the core wire 50 is the distal end 54 of the core wire, the first end of the tubular section is the distal end of the tubular section not represented in FIG. 8, and the second end of the core wire 50 is the proximal end 52 of the core wire.

In FIG. 8, the core wire is solid and does not have plurality of strands.

The retaining portion 70 has a cavity 71 on its side facing away from the proximal end of the tubular section and has a passage 74 extending axially from the cavity 71 through the retaining portion 70 to a side of the retaining portion facing the proximal end of the tubular section. Further, the distal end 54 of the core wire has a thickening 76 in the form of a bead. The cavity 71 is designed to receive the thickening 76 and the passage 74 is designed to retain axially the thickening in the cavity when the core wire is inserted in the passage.

A fifth embodiment of the steerable device is disclosed in FIG. 9, of which only a portion of the elongated element 12 and the arrangement of the plurality of the magnetic elements without the tubular section is represented, i.e. without the tube enveloping the plurality of magnetic elements.

The first end of the core wire 50, i.e. the distal end 54 of the core wire and the first end, i.e. the distal end, of the tubular section are arranged in this embodiment like in the fourth embodiment.

The fifth embodiment differs from the fourth embodiment mainly in that a second end 52 of the core wire opposed to the first end 54 is retained in a fixed manner in a further retaining portion 70a at a second end of the tubular section instead of being free. To this end, the second end 52 of the core wire has a further thickening 76a in the form of a bead. Further, the core wire 50 is deformable in an elastic manner to allow an axial displacement of the plurality of magnetic elements 30 relative to the core wire 50 when the device tip is bent.

The further retaining portion 70a is arranged similarly to retaining portion 70 present in the fourth embodiment with a further cavity 71a designed to receive the further thickening 76a and a further passage 74a designed to retain axially the thickening in the cavity when the core wire is inserted in the passage.

### LIST OF REFERENCE NUMBERS

steerable device 10
elongated element 12
distal end of the steerable device 14
distal end of the elongated element 16
device tip 20
tubular section 22
magnetic element 30
external envelope of elongated element 32
axial bore 40
conduit 46
bore edge 48
core wire 50
proximal end of the core wire 52
proximal end of the tubular section 53
distal end of the core wire 54
distal end of the tubular section 55
strands of the core wire 60a, 60b, 60c
retaining portion 70
cavity 71
first magnetic part, second magnetic part 72a, 72b
flat end of first magnetic part 74a
flat end of second magnetic part 74b
thickening 76
beads 76a, 76b
free end of first magnetic part 78a
free end of second magnetic part 78b
bevel 79
base angle α (alpha), α1, α2, α3
longitudinal axis of elongated element L
first, second, third pair of adjacent free ends 80, 82, 84

1. Steerable device for use inside of a mammalian body, comprising:
a flexible elongated element, elongated along a longitudinal axis and configured to be navigated through the mammalian body;
a flexible device tip arranged at the distal end of the elongated element and configured for allowing steering of the elongated element by way of an external magnetic field, the device tip comprising a tubular section and a plurality of permanent magnetic elements disposed in the tubular section end-to-end with adjacent ends of opposite polarity and their respective magnetic axes disposed along the longitudinal axis; wherein each magnetic element has an axial bore extending along its magnetic element longitudinal axis from a first axial end of the magnetic element to a second axial end of the magnetic element opposed to the first axial end, and the axial bores of the plurality of magnetic elements form an axial conduit through the plurality of magnetic elements;
a core wire extending in the conduit at least through two magnetic elements of the plurality of magnetic elements;
wherein first axial ends and second axial ends have a bevel to allow for movement of the magnetic elements against one another and bending of the device tip.

2. Steerable device according to claim 1, wherein the bevel is flat, as seen in a longitudinal section of the magnetic element, and forms a base angle with respect to the magnetic element longitudinal axis, wherein the bevel of an axial end and the bevel of a further axial end of a pair of adjacent ends of successive magnetic elements form a base angle and a further base angle, respectively, designed to allow bending of the device tip by a bending angle corresponding to the sum of the base angle and the further base angle.

3. Steerable device according to claim 2, wherein the bevel of a first pair of adjacent ends of successive magnetic elements has a same first base angle, the bevel of a second pair of adjacent ends of successive magnetic elements has a same second base angle, and the first base angle is different from the second base angle.

4. Steerable device according to claim 3, wherein the first pair of adjacent ends is arranged proximally to the second pair of adjacent ends and the first base angle is larger than the second base angle.

5. Steerable device according to claim 1, wherein the bevel is rounded, as seen in a longitudinal section of the magnetic element, to allow bending of the device tip.

6. Steerable device according to claim 5, wherein the bevel of a first pair of adjacent ends of magnetic elements has a same first curvature radius, the bevel of a second pair of adjacent ends of magnetic elements has a same second curvature radius, and the first curvature radius is different from the second curvature radius.

7. Steerable device according to claim 1, wherein the magnetic elements have the same axial length.

8. Steerable device according to claim 1, wherein a first end of the core wire is retained at a first end of the tubular section and a second end of the core wire opposed to the first end is arranged in the conduit and is free, to allow an axial displacement of the plurality of magnetic elements relative to the core wire when the device tip is bent.

9. Steerable device according to claim 8, wherein the first end of the core wire is the proximal end of the core wire, the first end of the tubular section is the proximal end of the tubular section and the second end of the core wire is the distal end of the core wire.

10. Steerable device according to claim 8, wherein the first end of the core wire is the distal end of the core wire, the first end of the tubular section is the distal end of the tubular section and the second end of the core wire is the proximal end of the core wire.

11. Steerable device according to claim 8, wherein the core wire is made of a plurality of strands extending parallel to each other along the longitudinal axis or made of a plurality of strands bundled along the longitudinal axis.

12. Steerable device according to claim 11, wherein the plurality of strands have each a length corresponding to a multiple of the length of the magnetic elements.

13. Steerable device according to claim 8, wherein the tubular section comprises a retaining portion arranged at the first end of the tubular section designed to retain the first end of the core wire at the first end of the tubular section.

14. Steerable device according to claim 13, wherein the retaining portion has a cavity on its side facing away from a second end of the tubular section and has a passage extending axially from the cavity through the retaining portion to a side of the retaining portion facing the second end of the tubular section, and in that the first end of the core wire has a thickening, wherein the cavity is designed to receive the thickening and the passage is designed to retain axially the thickening in the cavity when the core wire is inserted in the passage.

15. Steerable device according to claim 13, wherein the core wire is glued to the retaining portion or melted with the retaining portion.

16. Steerable device according to claim 1, wherein each magnetic element of the plurality of magnetic elements is made of two magnetic parts that are symmetrical to each other with respect to a symmetry plane cutting the magnetic axis perpendicularly at a middle point of the magnetic element as seen in the longitudinal direction, and are disposed end-to-end with adjacent ends of opposite polarity.

17. Steerable device for use inside of a mammalian body, comprising:
a flexible elongated element, elongated along a longitudinal axis and configured to be navigated through the mammalian body;
a flexible device tip arranged at the distal end of the elongated element and configured for allowing steering of the elongated element by way of an external magnetic field, the device tip comprising a tubular section and a plurality of permanent magnetic elements disposed in the tubular section end-to-end with adjacent ends of opposite polarity and their respective magnetic axes disposed along the longitudinal axis; wherein each magnetic element has an axial bore extending along its magnetic element longitudinal axis from a first axial end of the magnetic element to a second axial end of the magnetic element opposed to the first axial end, and the axial bores of the plurality of magnetic elements form an axial conduit through the plurality of magnetic elements;
a core wire extending in the conduit through the plurality of magnetic elements;
wherein a first end of the core wire is retained in a fixed manner at a first end of the tubular section and a second end of the core wire opposed to the first end is retained in a fixed manner at a second end of the tubular section, wherein the core wire is deformable in an elastic manner to allow an axial displacement of the plurality of magnetic elements relative to the core wire when the device tip is bent.

18. Steerable device for use inside of a mammalian body, comprising:
a flexible elongated element, elongated along a longitudinal axis and configured to be navigated through the mammalian body;
a flexible device tip arranged at the distal end of the elongated element and configured for allowing steering of the elongated element by way of an external magnetic field, the device tip comprising a tubular section and a plurality of permanent magnetic elements disposed in the tubular section end-to-end with adjacent ends of opposite polarity and their respective magnetic axes disposed along the longitudinal axis; wherein each magnetic element has an axial bore extending along its magnetic element longitudinal axis from a first axial end of the magnetic element to a second axial end of the magnetic element opposed to the first axial end, and the axial bores of the plurality of magnetic elements form an axial conduit through the plurality of magnetic elements;
a core wire extending in the conduit through the plurality of magnetic elements;
wherein a first end of the core wire is retained at a first end of the tubular section and a second end of the core wire opposed to the first end is retained at a second end of the tubular section, wherein the length of the core wire is longer than the axial length of the plurality of magnetic elements, wherein the first end of the core wire and/or the second end of the core wire are retained with a play designed to allow the axial displacement of the plurality of magnetic elements relative to the core wire when the device tip is bent.
